(19) United States
(12) Patent Application Publication
(10) Pub. No.: US 2014/0276616 A1
(43) Pub. Date: Sep. 18, 2014

(54) CATHETER-BASED DEVICES AND METHODS FOR IDENTIFYING SPECIFIC ANATOMICAL LANDMARKS OF THE HUMAN AORTIC VALVE

(71) Applicant: Syntheon Cardiology, LLC, Miami, FL (US)

(72) Inventors: Kevin W. Smith, Coral Gables, FL (US); Derek Dee Deville, Coral Gables, FL (US); Korey Kline, Miami, FL (US)

(73) Assignee: Syntheon Cardiology, LLC, Miami, FL (US)

(21) Appl. No.: 14/208,997

(22) Filed: Mar. 13, 2014

Related U.S. Application Data
(60) Provisional application No. 61/790,251, filed on Mar. 15, 2013.

(51) Int. Cl. A61M 25/09

(52) U.S. Cl. CPC A61M 25/09; A61M 2025/09166; A61M 2025/09175
USPC 604/510; 604/164.13

(57) ABSTRACT

A valve-plane-defining catheter includes a hollow flexible sheath having a distal end and defining a distal opening, a guidewire assembly, and three radiopaque valve-nadir markers. The guidewire assembly has a guidewire slidably disposed in the flexible sheath and having a distal end, three wire arms each having a terminating end and a proximal end offset from the terminating end and attached to the distal end of the guidewire to dispose the three wire arms approximately 120 degrees from one another about a circle defined by the terminating end of the three wire arms. The three radiopaque valve-nadir markers disposed respectively at the terminating end of each of the three wire arms, each of the valve-nadir markers being sized to fit within the distal opening.

### CROSS-REFERENCE TO RELATED APPLICATIONS

This application claims the priority, under 35 U.S.C. §119, of copending U.S. Provisional Patent Application No. 61/790,251, filed Mar. 15, 2013; the prior application is herewith incorporated by reference herein in its entirety.

### FIELD OF THE INVENTION

The present invention lies in the field of stents, stent grafts, replacement heart valve devices (including aortic, pulmonary, mitral and tricuspid), and methods and systems for implanting stents, stent grafts and replacement heart valve devices. In particular, the present invention provides catheter-based devices and methods for precisely identifying the location of specific anatomical landmarks of the aortic valve of a patient's heart for the proper surgical implantation of a replacement valve therein.

### BACKGROUND OF THE INVENTION

Medical and surgical implants are placed often in anatomic spaces where it is desirable for the implant to conform to the unique anatomy of the targeted anatomic space and to secure a seal therein, preferably without disturbing or distorting the unique anatomy of that targeted anatomic space. For example, endovascular implant stents or stent-grafts are used for the treatment of aneurysms (e.g., aortic) and other defects of the vascular structure. In another example, a replacement heart valve device can be used to repair a valve of the heart (e.g., aortic valve) that is failing and such a device is an effective treatment for severe stenosis (i.e., hardening, narrowing, or constricting) of the aortic valve, for example.

Depending on the specific application, a catheter may be used through a peripheral arteriotomy site to deploy an endograft implant or a replacement heart valve device into a specific site as a less invasive and less strenuous alternative to more complex surgical procedures. Such intended sites include, but are not limited to, the aortic valve annulus, ascending aorta, aortic arch, and thoracic or abdominal aorta. For example, with regard to aortic valve repair and/or replacement, the replacement valve assemblies may be deployed percutaneously into the aortic valve using a catheter-based delivery system. This type of minimally invasive procedure is particularly beneficial for patients who are not good surgical candidates for a variety of reasons, including being of high-risk for surviving open-heart surgery as a result of having other co-morbidities. Accordingly, this catheter-based approach opens the door for many patients to receive a life-saving replacement aortic valve device who, otherwise, would not be qualified to receive the replacement valve device by more conventional implantation methods. An exemplary inventive embodiment of such devices and methods are found in co-pending U.S. patent application Ser. No. 13/722,203, filed on Feb. 20, 2013, which application is incorporated herein in its entirety. This catheter-based approach is known in the art as Transcatheter Aortic-Valve Implantation (TAVI).

FIGS. 1 to 4 illustrate, in general, the progression of a standard TAVI system and procedure. Various features of the system and procedure are not shown in these figures for reasons of simplicity and clarity. In FIGS. 1 to 4, there is shown a diagrammatic representation of the arterial vascular network and heart of the upper body of a human being. In FIG. 1, a guide wire 110 of a catheter 100 of the system 1 is depicted as having already been inserted through the right iliac artery 20 and advanced all the way into the actual aortic valve 10 of the patient. The replacement aortic valve assembly 120 (only diagrammatically depicted) is disposed in the right iliac artery 20 in a collapsed and compressed state so that it may easily traverse the arterial network until it reaches the implantation site in the aortic valve. Turning to FIG. 2, the replacement aortic valve assembly 120 has now advanced to a position on the guide wire 110 that is within the abdominal aorta 30, adjacent the renal arteries 40. At this point in time, the replacement aortic valve assembly 120 is still in its collapsed state. Next, in FIG. 3, the replacement aortic valve assembly 120 has entered the aortic valve 10. As depicted in FIG. 4, once the replacement aortic valve assembly 120 has reached the implantation site within the actual aortic valve 10, the replacement aortic valve assembly 120 is expanded to assume the perimeter of the implant site such that it accommodates to the natural geometry of the implantation site. Thereafter, the guide wire 110 is retracted.

Despite numerous benefits and advantages of this minimally invasive catheter-based approach, there exist a number of limitations in currently-existing procedures when compared to the more surgically invasive methods such as open-heart surgery. For example, because a surgeon does not have a direct view of the aortic valve of the patient's heart using this catheter-based approach, it is more difficult to determine the precise and proper placement of the replacement aortic valve assembly within the existing aortic valve. As is clearly shown in FIGS. 1 to 4, a surgeon is unable to directly view the advancement of the replacement aortic valve assembly 112 through the arterial network (as described above) and, once the replacement aortic valve assembly 112 reaches its eventual position within the aortic valve 10, a surgeon does not have a direct view of the placement of the replacement aortic valve assembly within the aortic valve 10 of the heart. Rather, in combination with a surgeon's esteemed physiological knowledge and his or her well-practiced tactile skills as to how the proper advancement and implantation of the replacement aortic valve assembly should feel, a surgeon can only indirectly view, essentially from afar, the movement of the replacement aortic valve assembly through the arterial network and roughly approximate its placement within the aortic valve of the patient by injecting a radiopaque agent into the bloodstream (thereby creating a visible contrast of the blood flow on an angiographic image screen) and following the progression of the guide wire and the replacement aortic valve assembly on the screen. In particular, a desirable implant orientation aligns two planes with one another. The first plane is defined by the by the nadirs of each of the three cusps of the aortic valve to be replaced and is referred to herein as the native nadir plane. The second plane is defined by the nadirs of each of the three cusps of the aortic valve replacement device and is referred to herein as the implant nadir plane. As so defined, a most desirable implant orientation aligns the implant nadir plane to the native nadir plane.

Due to the inability of the surgeon to directly see and manipulate the heart structure and the replacement valve, it is increasingly difficult to place and implant (or attach) the replacement aortic valve assembly within the existing aortic valve and achieve co-planar alignment. Without proper placement, the effectiveness and functionality of the replacement valve may become greatly compromised and even dislodge into the aorta.

In addition to the inherent imprecision of this catheter-based approach, there is also a limit to the amount of radiopaque contrast agent that can be safely administered to a patient. Above a certain threshold concentration, radiopaque agents are known to be poisonous and can cause adverse reactions in a patient's bloodstream and tissues that, in some instances, can be life-threatening.

Accordingly, a need exists to overcome the problems with the prior art systems, designs, and processes for transcatheter implantation of a replacement valve device, such as an aortic valve replacement device.

### SUMMARY OF THE INVENTION

The present invention provides catheter-based surgical devices and methods for implanting a replacement valve device that overcome the hereinafore-mentioned disadvantages of the heretofore-known devices and methods of this general type and that provide such features with improvements that increase the ability of such an implant to be precisely positioned and to minimize the amount of injected contrast needed for visualization.

Specifically provided here are catheter-based surgical devices and methods for identifying and visualizing, in a substantially fool-proof manner, the precise locations of specific anatomical landmarks of an actual aortic valve of the heart of a patient. Thereafter, once these anatomical landmarks are precisely identified and visibly marked to define the native nadir plane, the surgeon can simply align or physically match the anatomical landmarks of the actual aortic valve of the patient with the corresponding structures of the replacement aortic valve device to ensure proper placement of the replacement valve device and co-planar alignment of the implant nadir plane and the native nadir plane.

Further provided are catheter-based surgical devices and methods for identifying and visualizing these specific anatomical landmarks of the actual aortic valve of any patient irrespective of the unique anatomy of that patient. While the existence of these specific anatomical landmarks of the aortic valve is common to all human beings, the precise location and particular structure of these anatomical landmarks vary amongst patients Like all parts of the body, the anatomy of the aortic valve is inherently unique from one patient to another and, in some instances, may have irregularities or defects that are either congenital or are a result of injury or disease. As described in further detail below, the inventive devices and methods described herein allow the precise physical contours of the patient's aortic valve to actually guide, or lead, in situ, the identification of the specific anatomical landmarks at focus herein and, therefore, the varying physiology from one patient to another does not affect the ability to identify the specific anatomical landmarks of the aortic valve that are particular to that patient. The devices and methods described herein do not rely on, or are not based upon, any pre-determined and predictive calculation, estimation, or data-averaging of where these specific anatomical landmarks should, or might, lie for any given patient.

With the foregoing and other objects in view, there is provided, in accordance with the invention, a method for defining a valve-plane of an aortic valve including the steps of guiding a distal end of a valve-plane-defining catheter at least through a portion of the aortic arch towards the aortic valve, the valve-plane-defining catheter having a flexible sheath, a guidewire assembly with a distal portion, and a guidewire, extending the distal portion of the guidewire assembly out from a distal end of the flexible sheath to expose three radiopaque valve-nadir markers of the distal portion disposed approximately 120 degrees from one another about a circle defined by the valve-nadir markers, and further extending the distal portion out from the sheath towards the aortic valve until the valve-nadir markers stop advancement by reaching respective ones of the aortic valve leaflet nadirs.

With the objects of the invention in view, there is also provided a valve-plane-defining catheter includes a hollow flexible sheath having a distal end and defining a distal opening, a guidewire assembly, and three radiopaque valve-nadir markers. The guidewire assembly has a guidewire slidably disposed in the flexible sheath and having a distal end, three wire arms each having a terminating end and a proximal end offset from the terminating end and attached to the distal end of the guidewire to dispose the three wire arms approximately 120 degrees from one another about a circle defined by the terminating end of the three wire arms. The three radiopaque valve-nadir markers disposed respectively at the terminating end of each of the three wire arms, each of the valve-nadir markers being sized to fit within the distal opening.

In accordance with another mode of the invention, the guiding and extending steps are carried out under fluoroscopy.

In accordance with a further feature of the invention, the valve-nadir markers are provided at the ends of three respective wire arms each attached at their respective proximal ends to the distal end of the guidewire.

In accordance with an added feature of the invention, the proximal end is radially offset from the terminating end.

In accordance with an additional feature of the invention, the proximal end is radially inwardly offset from the terminating end.

In accordance with a concomitant feature of the invention, the three wire arms each have a different length to position the three valve-nadir markers in line with one another when the three valve-nadir markers are disposed in the distal end of the sheath.

Additional advantages and other features characteristic of the systems and methods describe herein will be set forth in the detailed description which follows and may be apparent from the detailed description or may be learned by practice of exemplary embodiments of the invention. Although the inventive systems and methods are illustrated and described herein as being devices and methods for precisely identifying the location of specific anatomical landmarks of an actual aortic valve of a patient's heart for the proper surgical implantation of a replacement valve device therein, it is, nevertheless, not intended to be limited to the details shown because various modifications and structural changes may be made therein without departing from the spirit of the invention and within the scope and range of equivalents. Additionally, well-known elements of exemplary embodiments described herein will not be described in detail or will be omitted so as not to obscure the relevant details thereof.

Other features that are considered as characteristic are set forth below.

### BRIEF DESCRIPTION OF THE DRAWINGS

The accompanying figures, where like reference numerals refer to identical or functionally similar elements throughout the separate views, which may not be true to scale, and which, together with the detailed description below, are incorporated in and form part of the specification, serve to further illustrate various embodiments and to explain various principles and advantages all in accordance with the present invention. Advantages of embodiments described herein will be apparent from the following detailed description of the exemplary embodiments thereof, which description should be considered in conjunction with the accompanying drawings in which:

FIG. 1 is a fragmentary, perspective view of a prior art replacement aortic valve device in the right iliac artery in a process of being implanted, the replacement aortic valve device being in a collapsed, compressed state;

FIG. 2 is a fragmentary, perspective view of the replacement aortic valve device of FIG. 1 in the abdominal aorta in the process of being implanted;

FIG. 3 is a fragmentary, perspective view of the replacement aortic valve device of FIGS. 1 and 2 adjacent the aortic valve implantation site in the process of being implanted;

FIG. 4 is a fragmentary, perspective view of the replacement aortic valve device of FIGS. 1 to 3 implanted in the heart, the replacement aortic valve device being in an expanded state;

FIG. 5 is a front, elevational view of an exemplary embodiment of a catheter and guide wire assembly with the guide wire of the assembly not present;

FIG. 6 is a fragmentary, side perspective view of a distal end of the catheter of FIG. 5;

FIG. 7 is a fragmentary, side perspective view of the distal end of the catheter of FIGS. 5 and 6, the guide wire of the assembly having been inserted into the catheter and partially protruding out of the catheter to a first position;

FIG. 8 is a fragmentary, side perspective view of the distal end of the catheter of FIGS. 5 to 7, the guide wire partially protruding out of the catheter to a further, second position;

FIG. 9 is a fragmentary, side perspective view of the distal end of the catheter of FIGS. 5 to 8, the guide wire partially protruding out of the catheter to yet a further, third position;

FIG. 10 is a fragmentary, front perspective view of the distal end of the catheter of FIGS. 5 to 9, whereby the guide wire is partially protruding out of the catheter to an even further, fourth position;

FIG. 11 is a fragmentary, front elevational view of an initial step of an exemplary embodiment of an inventive method, the guide wire of the catheter and the guide wire assembly of FIGS. 1 to 10 having been introduced into an artificial rendering of a human aorta and advanced into the aortic valve;

FIG. 12 is a fragmentary, front elevational view of a subsequent step of the method of FIG. 11, the catheter of the catheter and guide wire assembly of FIGS. 1 to 10 having been inserted over the guide wire and introduced into the aorta and advanced into the abdominal aorta;

FIG. 13 is a fragmentary, front elevational view of a subsequent step of the method of FIGS. 11 and 12, the catheter having advanced into the aortic arch;

FIG. 14 is a fragmentary, side perspective view of a subsequent step of the method of FIGS. 11 to 13, the catheter having advanced into the ascending aorta;

FIG. 15 is a fragmentary, side perspective view of a subsequent step of the method of FIGS. 11 to 14, the catheter entering the sinuses of the aortic valve;

FIG. 16 is a fragmentary, side perspective view of a subsequent step of the method of FIGS. 11 to 15, the guide wire having been partially retracted from the distal end of the catheter and the catheter starting to approximate its measuring shape;

FIG. 17 is a fragmentary, side perspective view of a subsequent step of the method of FIGS. 11 to 16, the guide wire having been further retracted from the distal end of the catheter and the catheter continuing to approximate its measuring shape;

FIG. 18 is a fragmentary, side perspective view of a subsequent step of the method of FIGS. 11 to 17, the guide wire having been completely retracted from the distal end of the catheter and the catheter being substantially at its measuring shape;

FIG. 19 is a fragmentary, side perspective view of a subsequent step of the method of FIGS. 11 to 18, the distal end of the catheter being substantially at its measuring shape and seating at the commissure points of the aortic valve;

FIG. 20 is a fragmentary, side perspective view of another exemplary embodiment of an inventive catheter and guide wire assembly in a slightly actuated configuration;

FIG. 21 is a fragmentary, perspective view of the assembly of FIG. 20 with the assembly in a partially actuated configuration;

FIG. 22 is a fragmentary, side perspective view of the assembly of FIGS. 20 and 21 with the assembly in a further partially actuated configuration;

FIG. 23 is a fragmentary, side perspective view of the assembly of FIGS. 20 to 22 with the assembly in a fully actuated configuration;

FIG. 24 is a fragmentary, front perspective view of the assembly of FIG. 23;

FIG. 25 is a top plan view of an exemplary embodiment of an inventive mandrel being used to shape the three wire arms of the guide wire of the assembly of FIGS. 20 to 24;

FIG. 26 is a side, elevational view of the catheter and guide wire assembly of FIGS. 20 to 25 with the assembly in the slightly actuated configuration shown in FIG. 20;

FIG. 27 is a side, elevational view of the assembly of FIGS. 20 to 26 with the assembly in the fully actuated configuration shown in FIG. 23;

FIG. 28 is a fragmentary, front elevational view of an initial step of an exemplary embodiment of an inventive method with the catheter and guide wire assembly of FIGS. 20 to 27, in its unactuated configuration, having been introduced into an artificial rendering of the human aorta and advanced into the abdominal aorta;

FIG. 29 is a fragmentary, front elevational view of a subsequent step of the method of FIG. 28 with the assembly advanced into the aortic arch;

FIG. 30 is a fragmentary, front perspective view of a subsequent step of the method of FIGS. 28 and 29 with the assembly advanced into the ascending aorta;

FIG. 31 is a fragmentary, front perspective view of a subsequent step of the method of FIGS. 28 to 30 with the assembly in its partially actuated configuration to partially advance the guide wire out of the catheter and into the aortic sinuses of the aortic valve; and

FIG. 32 is a fragmentary, front perspective view of a subsequent step of the method of FIGS. 28 to 31 with the assembly in its fully actuated configuration to advance the guide wire into the aortic valve to a point where radiopaque ends of the catheter are within the nadirs of the cusps of the aortic valve.

### DETAILED DESCRIPTION OF THE INVENTION

As required, detailed embodiments are disclosed herein; however, it is to be understood that the disclosed embodiments are merely exemplary of the systems and methods, which can be embodied in various forms. Therefore, specific structural and functional details disclosed herein are not to be interpreted as limiting, but merely as a basis for any claims and as a representative basis for teaching one skilled in the art to variously employ the systems and methods described herein in virtually any appropriately detailed structure. Further, the terms and phrases used herein are not intended to be limiting; but rather, to provide an understandable description of the invention. While the specification may conclude with claims defining the features of the invention that are regarded as novel, it is believed that the invention will be better understood from a consideration of the following description in conjunction with the drawing figures, in which like reference numerals are carried forward.

Before the inventive aspects are disclosed and described, it is to be understood that the terminology used herein is for the purpose of describing particular embodiments only and is not intended to be limiting. The terms “a” or “an”, as used herein, are defined as one or more than one. The term “plurality”, as used herein, is defined as two or more than two. The term “another”, as used herein, is defined as at least a second or more. The terms “including” and/or “having”, as used herein, are defined as comprising (i.e., open language). The term “coupled,” as used herein, is defined as connected, although not necessarily directly, and not necessarily mechanically. Relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure. Described now are exemplary embodiments.

Referring now to the figures of the drawings in detail and first, particularly to FIGS. 5 to 10, there is shown a first exemplary embodiment of a catheter and guide wire assembly 200 for use in assisting the deployment of a transcatheter replacement aortic valve device (not shown). Even though this exemplary embodiment is illustrated as an assembly for use in deploying a replacement aortic valve device without the presence of a replacement aortic valve device, this embodiment is not to be considered as limited thereto. The catheter and guide wire assembly 200 disclosed herein can be used in a procedure in which it is desired to precisely identify the locations of the valve leaflet commisures of an aortic valve of a patient, such as in balloon valvuloplasty.

The catheter and guide wire assembly 200 is comprised of a catheter 210 and a guide wire 220. In FIG. 5, the catheter 210 of the assembly 200 is shown, without the presence of the guide wire 220. The catheter 210 is comprised of a substantially straight, flexible sheath 230 having an interior lumen therethrough (not shown). The catheter sheath 230 may be comprised of any material and of any size/diameter that is suitable for introduction of the catheter 210, percutaneously, into vascular of the human body (e.g., the femoral artery) and running the catheter 210 through the arterial network of the body, as well as allowing a guide wire and a compressed replacement aortic valve device to be inserted therethrough. In addition, portions of the catheter sheath 230 are comprised of a radiopaque material so that the catheter 210 can be easily seen on an X-ray or angiographic image. For example, in this particular exemplary embodiment, the catheter sheath 230 is a 5-French sheath and is comprised of a Polytetrafluoroethylene (PTFE) liner with Polyurethane (PU) or a similar outer jacket. As an alternative, an olefinic material can be used.

The catheter sheath 230 has a proximal end 240 and a distal end 250 and is shaped to be removably inserted into and to be removed from the vasculature of the human body. The distal end 250 of the catheter sheath 230 terminates at a distal tip portion 260. As shown in closer detail in FIG. 6, this distal tip portion 260 is heat-set to form and to flexibly maintain the shape of nearly a full-turn loop 270. In an exemplary embodiment, the loop circumscribes greater than 300 degrees, specifically, greater than 270 degrees, and, in particular, greater than 180 degrees. The tip of the loop 270 can be formed to curve back toward the main body of the catheter and prevent catching on the native leaflets (not illustrated). The plane of the loop 270 is as flat as possible and is as close to perpendicular as possible. The loop 270 has a number of holes cut in it to allow the contrast injected to be evenly dispersed as is normally done in a pig tail catheter. These holes can be disposed all the way around the loop 270 or can be disposed partially around the loop 270 with the number of holes and their position optimized to balance the flow from the tip and through the holes evenly in the aorta area.

To complete the catheter and guide wire assembly 200, a guide wire 220 is inserted into and through the length of the interior lumen of the catheter sheath 230 to a point at which the relative structural strength of the guide wire 200, in comparison to the structural strength of the catheter sheath 230, causes the terminating loop 270 of the distal tip portion 260 of the catheter sheath 230 to substantially straighten while the guide wire 220 is at least partially disposed in the distal tip 260. In the particular exemplary embodiment shown in FIGS. 5 and 6, to substantially straighten the loop 270 of the distal tip 260, the guide wire 220 traverses the entire length of the catheter 210 and protrudes out a distance from the distal tip 260. This position of the guide wire 220 is shown in FIG. 10. In this position, the guide wire 220 is approximately three to four inches outside the tip 260 of the catheter sheath 230. FIGS. 7, 8, and 9 illustrate the gradual advancement of the guide wire 220 through the distal end 250 of the catheter sheath 230 and the incremental straightening of the distal tip 260 resulting from the guide wire 220 moving through and out of the distal tip 260. The guide wire 220 may be comprised of any radiopaque material that can be easily seen on an X-ray or angiographic image. In addition, the guide wire 220 may be of any size/diameter that is suitable for inserting the guide wire through the interior lumen of the catheter sheath 230. For example, in this particular exemplary embodiment, the guide wire 220 is a 0.035″ wire that is comprised of Nitinol or stainless steel or similar material.

Referring now to FIGS. 11 to 19, there is shown an exemplary embodiment of a surgical method for operating the exemplary device of the embodiment shown in FIGS. 5 to 10 to precisely identify and visualize the commissure points of an actual aortic valve of a patient for purposes of determining the proper placement of a replacement aortic valve device. For purposes of illustrating this exemplary method, there is shown an artificial scale rendering of the aortic valve and the aorta and a portion of its principle branches in the upper portion of the human body using a transparent tubing network 300. The branching structures 310, 312 represent the right deep and left femoral arteries and the left internal, left common, and external iliac arteries. Structure 320 represents the abdominal aorta. The branching structures 330, 332 represent the mesenteric, renal, ulnar, gastric, and hepatic arteries. Structure 340 represents the thoracic aorta. Structures 350 and 360 represent the aortic arch and the ascending aorta, respectively, leading into the aortic valve 370 of the heart 380. The portion of the tubular structure 300 that represents the aortic valve 370 is shown in closer detail in FIGS. 14 to 19.

A human aortic valve is known as a semilunar (SL) valve because it is comprised of three crescent moon-shaped cusps or “leaflets.” These cusps are referred to as the left, right, and posterior cusps. These cusps or leaflets of the aortic valve 370 are shown as pocket-like structures 390 in the artificial representation of the valve shown in FIGS. 11 to 19. In operation, the aortic valve allows blood to be ejected from the heart but prevents backflow of blood into the ventricles. The free borders of the cusps project into the lumen of the artery. Pressure builds up within the chambers of the heart when the ventricles contract. Once the pressure in the ventricles exceeds the pressure in the arteries, the aortic valve opens and permits ejection of blood from the ventricles and into the pulmonary trunk and aorta. As the ventricles relax, blood starts to flow back towards the heart. As the back-flowing blood fills the cusps, the aortic valve closes.

Associated with each cusp is a small dilation of the proximal aorta. These areas are referred to as the aortic sinuses. Each cusp attaches to the wall of the aorta by its convex outer margin. The level at which this attachment occurs is referred to as the sinotubular junction. A line of demarcation known as the supraaortic ridge identifies the sinotubular junction and is essentially a thickened aortic wall. The small spaces between the attachment points of each cusp are called the aortic valve commissures. These three commis sures lie at the apex of the annulus of the aortic valve and are composed of collagenous fibers oriented in a radial fashion, spaced approximately 120° degrees apart. The commissures provide support for the valvular structures and allow stress on the valve cusps to be transmitted into the aortic wall. The bottom or lowermost point of the “belly” area of each cusp or leaflet of the aortic valve is referred to as the nadir or nadir point. Thus, the nadir points are also spaced approximately 120° degrees apart and, together, form a circular ring that defines the plane of the aortic valve annulus, which is the goal for best implantation. The approximate positions of the commissure point, nadir point, and aortic sinus of each visible cusp are shown at 400, 410, and 440, respectively.

Because the commissure points 400 constitute the natural physiological points of attachment between the aortic valve and the aortic wall, it is desirable that any replacement aortic valve device be positioned such that the replacement leaflets function in a manner similar to the native anatomy. Therefore, in determining the proper placement of the replacement aortic valve, it is important to locate the commis sure points 400 as part of the implantation procedure.

Referring back to FIG. 11, there is shown an initial procedural step of this first exemplary embodiment of a surgical method of operating the exemplary catheter and guide wire assembly 200 of the embodiment shown in FIGS. 5 to 10. In this initial step, using any appropriate procedure known in the art, a surgeon accesses a peripheral artery of the patient for introduction of just the guide wire 220 portion of the catheter and the guide wire assembly 200 into the vasculature of the patient. For example, in this particular exemplary embodiment, the guide wire 220 is introduced into the femoral (or iliac) artery 310 of the patient. Thereafter, by following the path of the radiopaque guide wire 220 using X-ray or angiographic imagery, the surgeon inserts the guide wire 220 up through the abdominal aorta 320, then into the thoracic aorta 340, about the aortic arch 350, down into the ascending aorta 360, through the aortic valve 370, and into the left ventricle of the heart 380. Next, as depicted in FIG. 12, the sheath 230 of the catheter 210 of the assembly 200 is introduced over the guide wire 220 at the same point of entry. Accordingly, in this particular exemplary embodiment, the catheter sheath 230 is guided onto the guide wire 220 through the femoral (or iliac) artery 310, as well, and is advanced into the thoracic aorta 340. As shown in FIG. 12, the loop 270 (which has been previously set into the distal tip 260 of the catheter sheath 230) has been almost completely straightened by the guide wire 230 running therethrough. In this substantially straight configuration, the catheter sheath 230 is easily guided along the guide wire 220 and smoothly traverses the arterial network without causing any obstruction that would otherwise occur if the loop 270 were still present. In FIG. 13, the catheter sheath 230 has further advanced into the aortic arch 350. In FIG. 14, the catheter sheath 230 has journeyed past the ascending aorta 360 and approaches the aortic valve 370 without passing through the opening of the three cusps or leaflets 390 of the valve.

At this point in the procedure, the terminating tip 260 of the distal end 250 of the catheter sheath 230 is still being maintained in its substantially straight configuration by the traversing guide wire 220. However, once the catheter sheath 230 has reached the aortic valve 370, the guide wire 220 is incrementally retracted backwards from inside the distal end 250 of the catheter sheath 230. FIGS. 15, 16, and 17 show the effect of the guide wire 220 being gradually retracted backwards and out of the aortic valve while still leaving the distal tip 260 of the catheter sheath 230 in the aortic valve. As a result, the distal tip 260 incrementally springs back into its initial form of the loop 270, due to the absence of the guide wire 220, which causes the distal tip 260 of the catheter sheath 230 to coil just downstream of the aortic valve 370 and lie in the aorta 360 at a position adjacent and above the commisures 400 of the aortic valve 370. In FIG. 18, the guide wire 220 has been completely retracted out of the distal tip 260 of the catheter sheath 230 and the tip 260 has substantially reconstituted into its loop shape as the loop 270 lies adjacent the aortic valve 370 in the aorta 360. In a final step, depicted in FIG. 19, longitudinal pressure is applied by the surgeon to the proximal end 240 (not shown) of the catheter sheath 230, thereby forcibly pressing the loop 270 of the distal tip 260 of the catheter sheath 230 towards and against the natural geometry of the aortic valve 370 until the loop 270 comes to rest and forms a circular and substantially perpendicular plane. At this point in time, due to the natural landscape of the aortic valve (as described in detail above), the looped tip 260 of the catheter sheath 230 has hit the three ridges formed by the commissure points 400 of the three leaflets 390 of the aortic valve and, as a result of this obstruction, defines a plane that can be seen and recorded. This plane forms at the precise locations of the commis sure points 400 of the aortic valve 370. Due to the radiopacity of the loop 270 (which can be solid throughout or just points along the loop 270), the surgeon can easily see the exact position of the plane on an X-ray or angiographic image. Accordingly, the surgeon can visibly mark the precise locations of the commissure points 400 by looking at the plane (which signifies the presence of the commissure points by its exact formation) when the catheter 210 is in the position shown in FIG. 19. The surgeon is then able to use this visible marking created by the plane as a reference tool showing the precise location in which the replacement aortic valve device should be implanted.

By using this simple and inventive procedure just described, at no point does the surgeon need to visually approximate the location of the commissure points using the prior art methods of injecting a contrast dye into a patient's bloodstream and following the fluid and/or dynamic activity of the dye on the angiographic image to try to perceive the anatomy of the patient's aortic valve.

The replacement valve body needs to seal to the native annulus, which is, in part, defined by the nadirs of the valve and follows a three-dimensional curve that rises and falls in a pattern that matches the native leaflets and, therefore, the commissures. Once this described catheter is in place and resting on the native commissures and expanded into the valve sinus, its axial position will be fixed. From here, after an angiogram, the distance to the nadirs will be known and they will follow a plane parallel to the plane of the catheter loop but offset by the leaflet height. Therefore, the described catheter will be a fixed landmark for the position and plane in which the replacement valve should be deployed. The transcatheter valve can be passed through the loop 270 of this catheter as it is passed through the native valve. The landmarks of the valve can be compared to the loop 270 and to the known distance to the nadirs (and, therefore, the native annulus) and precise longitudinal positioning can be accomplished.

However, location of the downstream ends of the natural commisures does not give the surgeon the most ideal plane for implanting the replacement valve assembly because the height of the commisures in the aortic valve can be greater or smaller than the height of the commisures in the replacement aortic valve. In such a case, the replacement valve leaflets could be offset from the natural valve leaflets, which is undesirable. Accordingly, being able to define the plane of the leaflet bottoms would be beneficial and is provided in the following exemplary embodiments of the invention.

Referring now first to FIGS. 26 and 27 and then to FIGS. 20 to 24, there is shown another exemplary embodiment of a catheter and guide wire assembly 500 for use in deploying a replacement aortic valve device (not shown). In contrast to the exemplary embodiment of the catheter and guide wire assembly 200 discussed above and shown in FIGS. 5 to 10, the assembly 500 of this second exemplary embodiment is not used to determine the precise locations of the commissure points 400 of the human aortic valve but, rather, is used to determine the precise locations of the nadir points 410 of the aortic valve. Even though this exemplary embodiment is illustrated as an assembly for use in assisting deployment of a replacement aortic valve device without the presence of a replacement aortic valve device, this embodiment is not to be considered as limited thereto. The catheter and guide wire assembly 500 disclosed herein can be used in any procedure in which it is desired to precisely identify the locations of the nadir points of the aortic valve of a patient, such as in balloon valvuloplasty.

In this exemplary embodiment, the catheter and guide wire assembly 500 is comprised of a catheter 510 and a guide wire assembly 520. The catheter 510 is comprised of a substantially straight, flexible sheath 530 having an interior lumen therethrough. The catheter sheath 530 may be comprised of any material and be of any size/diameter that is suitable for introduction of the catheter 510, percutaneously, into a major artery of the human body (e.g., femoral artery) and running the catheter 510 through the arterial network of the body, as well as allowing the guide wire assembly 520 described below and a compressed replacement aortic valve device to be inserted therethrough. For example, in this particular exemplary embodiment, the catheter sheath 530 is a 6-French sheath and is comprised of a PTFE liner with a PU or similar outer jacket; an alternative to this being an olefinic material. Or, the outer jacket can be all of urethane if a lubricious coated wire is passing therethrough. In addition, the catheter sheath 530 has at least some radiopaque markers so that the catheter 510 can be easily seen on an X-ray or angiographic image. The catheter sheath 530 has a proximal end 540 and a distal end 550 and is shaped to be removably inserted into and to be removed from the vasculature of the human body.

The guide wire assembly 520 is comprised of a guide wire 620 and, at the distal end 524 of the guide wire, a three-pronged extension wire 560 that is operatively attached to the distal end 524. The guide wire assembly 520 may be comprised of any radiopaque material that can be easily seen on an X-ray or angiographic image. In addition, the guide wire 620 may be of any size/diameter that is suitable for inserting the guide wire 620 through the interior lumen of the catheter sheath 530. For example, in this particular exemplary embodiment, the guide wire 620 is a 0.025″ wire that is comprised of stainless steel. As best shown in FIG. 24, the guide wire 620 terminates into the three-pronged extension wire 560. The three-pronged extension wire is comprised of three wire arms 570 of equal length (but they do not necessarily need to be of equal length). Each wire arm 570 is pre-constructed to have a slope or curve at an intermediate portion of its length such that each arm, when commonly attached to the distal end 524 of the guide wire 620, radiates outward from this common attachment point in a bloom-like manner. This inward slope or curve in each wire arm 570 also allows the wire arm to bend in inwardly if a compressive pressure is being applied at the terminating end 580 of the wire arm in a direction that is perpendicular to the longitudinal plane of the arm. When in this radiating configuration (as shown in FIGS. 24 and 27), each terminating end 580 of each wire arm 570 is equidistantly spaced approximately 120° degrees apart from the adjacent terminating ends 580 of the two other wire arms 570. In FIG. 25, there is shown one exemplary embodiment of a mandrel device 630 being used to create the desired slope or curve in each of the wire arms 570. The wire arms 570 may be comprised of any radiopaque material that can be easily seen on an X-ray or angiographic image. With respect to the size/diameter of the wire arms 570, the chosen gauge must provide a certain amount of rigidity and collinear strength in order for the wire arms 570 to hold their radiating shape and not collapse on one another when collectively attached to the distal end 524 of the guide wire 620. In addition, the chosen gauge for the wire must also allow the wire arms 570 to substantially return to their radiating shape after instances where the wire arms 570 have been compressed against one another and are momentarily constrained in a tight space. For example, as described in detail below and shown in FIG. 20, when the catheter sheath 530 is initially inserted into the patient, the guide wire assembly 520 has already been inserted through the length of the catheter sheath 530 such that the wire arms 570 of the three-pronged extension wire 560 are fully contained and compressed together inside the distal end 550 of the catheter sheath 530. However, once the assembly 500 has reached its destination point at the aortic valve, the guide wire assembly 520 is partially advanced out of the catheter sheath 530 as shown in FIGS. 21 to 24. Eventually, as shown in FIGS. 23, 24, and 27, the three-pronged extension wire 560 fully exits the distal end 550 of the catheter sheath 530 and the wire arms 570, having now been freed, resume their radiating, bloom-like configuration. This gradual exit of the three-pronged extension wire 560 from the catheter sheath 530 and the outward spring of the wire arms 570 once they are completely free is illustrated in the progression shown from FIG. 21 to FIG. 24. Accordingly, the gauge chosen for the wire arms 570 allows for the arms to be temporarily compressed together while inside the catheter sheath 530 while also allowing the arms to substantially return to their desired radiating shape after having been constrained.

At each terminating end 580 of each wire arm 570, there is a radiopaque body 590. For a specific purpose that is described in detail below, the bodies 590 are configured to collectively form three points necessary to define a plane, where each body 590 is separated from the two adjacent bodies 590 by a pre-determined 120° degrees. Accordingly, when the three-pronged extension wire 560 is in the radiating configuration shown in FIG. 24, the relative positions of the bodies 590 with respect to one another mimic the approximate positions of the three nadir points 410 of the human aortic valve. When in the constrained position that was described above in reference to FIG. 20, the collective diameter of the three bodies 590 is larger than the inner diameter of the interior lumen of the catheter sheath 530. Accordingly, the three bodies 590 are caught in a tight cluster 600 just outside the distal opening 610 of the catheter sheath 530. This is the most compact configuration that the catheter and guide wire assembly 500 can be placed in when the extension wires 560 are of equal length. Thus, this is the configuration that the assembly has when it is inserted into the vasculature of the patient in order that the assembly 500 can easily pass through the arterial network and into the aortic valve without causing any obstruction to occur along its path.

In this particular exemplary embodiment, the bodies 590 are in the shape of solid metal spheres having a diameter of 0.040″ and are comprised of stainless steel. However, this spherical shape is just one example of a variety of body shapes that are suitable for use in the described methods and systems. Bodies 590 may be comprised of any radiopaque material (such as tungsten or tantalum) and can be of any 3-dimensional body shape that will adequately appear on an X-ray or angiographic image and will not obstruct the arterial network when held in the cluster 600 as described above. In particular, a spherical shape presents a blunt end to the nadir and prevents damage by penetration.

The configuration of the three-pronged wire extension 560 described above is just one illustration of a number of conceivable embodiments that are contemplated by the systems and methods described herein. For example, to obviate the bodies 590 forming a cluster 600 at the distal opening 610 of the collar of the catheter sheath 530, the wire arms 570 could be constructed to each have a different length, one longer than the other, such that, when compressed together within the catheter sheath 530 as shown in FIG. 20, the bodies 590 will line up longitudinally in a slimmer, staggered fashion to fit inside the catheter sheath 530 if appropriately sized for such entry. Also notches at these bodies 590 can create passages for the wires, further reducing the maximum diameter to that of just the bodies 590.

Referring now to FIGS. 26 to 32, there is shown an exemplary embodiment of a surgical method for operating the exemplary device of the embodiment shown in FIGS. 20 to 24 to precisely identify and visualize the nadir points of an actual aortic valve of a patient for purposes of determining the proper placement of a replacement aortic valve device. To illustrate this process, there is again an artificial rendering of the aortic valve and the aorta and a portion of its principle branches in the upper portion of the human body using the transparent tubing network 300.

FIG. 28 depicts an initial step whereby, using any appropriate procedure known in the art, a surgeon accesses a peripheral artery of the patient for introduction of the entire catheter and guide wire assembly 500 into the vasculature of the patient. At this point, the assembly 500 is in its most compacted configuration (as shown in FIGS. 20 and 26), referred to herein as an “unactuated” configuration. For example, in this particular exemplary embodiment, the assembly 500 is introduced into the femoral (or iliac) artery 310 of the patient. The guide wire assembly 520 has been inserted through the length of the catheter sheath 530 and the arms 570 of the three-pronged extension wire 560 are fully contained inside the distal end 550 of the catheter sheath 530. The bodies 590 are shown as remaining exposed in a tight cluster 600 just outside the distal opening 610 of the catheter sheath 530. Alternatively, they can be fully retracted into the sheath 530.

Thereafter, by following the path of the radiopaque assembly 500 using X-ray or angiographic imagery, the surgeon advances the assembly 500 into and past the thoracic aorta 340, shown in FIG. 29. As depicted in FIG. 30, the assembly 500 is advanced past the aortic arch 350 and enters the ascending aorta 360. At this point, the assembly 500 is just about to enter the aortic sinuses 440 and, as an assembly, is not advanced any further. Instead, the surgeon continues to apply pressure just to the guide wire assembly 520 portion of the device; in other words, the catheter sheath 530 stays in place. This movement causes the three-pronged extension wire 560 to exit the distal end 550 of the catheter sheath 530 and to cause the wire arms 570 to begin radiating outward, as shown in FIG. 31. Accordingly, the three-pronged wire extension 560 is in a partially “actuated” configuration. As the surgeon continues to advance the guide wire assembly 520, a slight rotation of the guide wire assembly 520 allows the bodies 590 to become naturally oriented to enter into each aortic sinus 440 of the three cusps 390 of the aortic valve 370 due to the natural geometry of the aortic valve. This position is shown in FIG. 32. At this point, the three-pronged wire extension 560 is in a fully actuated configuration and can be advanced until each body 590 rests in the nadir of each valve leaflet. This same progression of the three-pronged extension wire 560 exiting from the catheter sheath 530 is shown in FIGS. 21, 22 and 23, the only difference being that, in those views, the assembly 500 is not being used in a (simulated) patient.

Once the guide wire 520 has advanced into the aortic valve 370 to a point where the bodies 590 reach a dead end within the aortic cusps 390 so that they cannot advance any further despite any continued rotation of the guide wire assembly 520, the bodies 590 have settled into the bottom or lowermost points 410 of the three cusps 390, i.e., the nadir points. Due to the radiopacity of the bodies 590, the surgeon can easily see the bodies 590 on an X-ray or angiographic image. Accordingly, the surgeon can visibly mark the precise locations of the nadir points 410 by looking at the bodies 590 (which signify the presence of the nadir points by their resting places) when the assembly is in the position shown in FIG. 32. An exemplary marking of the three bodies 590 is depicted in FIG. 32 with dashed circles. The surgeon is, then, able to use these three markings created by the bodies 590 as a reference tool showing the precise location in which the replacement aortic valve device should be implanted. During the implantation procedure, the replacement aortic valve device can simply be aligned with the reference markings provided by the bodies 590 to determine the replacement valve's precise placement.

Each of the bodies 590 can be on its own wire running all the way to the proximal end of the catheter and can have a longitudinal compression spring or other similar mechanism that allows them to stroke through some longitudinal distance. Such an assembly would keep the bodies 590 engaged with the nadir points and free each body 590 to assume a point on the plane of the native annulus. This would also reduce the need of the operator to keep the catheter in a fixed position. As long as forward pressure is being applied, the bodies 590 will be in the correct position.

Additionally, the catheter body can be formed with a sufficient recurve to make it coaxial with the native annulus once it is passed over the arch. Further, the wires 560 can be configured to have one marking body 590 extend coaxially with the catheter and the other two bodies 590 to expand away from the first. Such a configuration would allow the catheter to be biased toward the outside of the arch, which is most easily accomplished and gives a more predictable positioning given that it is being forced against native anatomy. A further advantage of this configuration is that the marker catheter will be out of the way for any introduction of the replacement valve.

The described systems and processes provide a great advantage over the use of a captured angiographic image that is overlayed or compared to the current fluoroscopic image. Any deviations or slight changes in position of the native anatomy will not be taken into account with overlays or digital wireframes. In comparison, the markers of the catheters described herein will be continuously visible and will give positive and definitive feedback of the location and plane of the native annulus and, thus, of the target implantation site. With this information, the replacement valve can be advanced utilizing this marker catheter and through the native valve and be expanded in place with confidence that the position and orientation at the time of implantation are directly being matched to the native anatomy.

It is noted that various individual features of the inventive processes and systems may be described only in one exemplary embodiment herein. The particular choice for description herein with regard to a single exemplary embodiment is not to be taken as a limitation that the particular feature is only applicable to the embodiment in which it is described. All features described herein are equally applicable to, additive, or interchangeable with any or all of the other exemplary embodiments described herein and in any combination or grouping or arrangement. In particular, use of a single reference numeral herein to illustrate, define, or describe a particular feature does not mean that the feature cannot be associated or equated to another feature in another drawing figure or description. Further, where two or more reference numerals are used in the figures or in the drawings, this should not be construed as being limited to only those embodiments or features, they are equally applicable to similar features or not a reference numeral is used or another reference numeral is omitted.

The phrase “at least one of A and B” is used herein and/or in the following claims, where A and B are variables indicating a particular object or attribute. When used, this phrase is intended to and is hereby defined as a choice of A or B or both A and B, which is similar to the phrase “and/or”. Where more than two variables are present in such a phrase, this phrase is hereby defined as including only one of the variables, any one of the variables, any combination of any of the variables, and all of the variables.

The foregoing description and accompanying drawings illustrate the principles, exemplary embodiments, and modes of operation of the invention. However, the invention should not be construed as being limited to the particular embodiments discussed above. Additional variations of the embodiments discussed above will be appreciated by those skilled in the art and the above-described embodiments should be regarded as illustrative rather than restrictive. Accordingly, it should be appreciated that variations to those embodiments can be made by those skilled in the art without departing from the scope of the invention as defined by the following claims.

What is claimed is:

1. A method for defining a valve-plane of an aortic valve, comprising:
guiding a distal end of a valve-plane-defining catheter at least through a portion of the aortic arch towards the aortic valve, the valve-plane-defining catheter having a flexible sheath, a guidewire assembly with a distal portion, and a guidewire;
extending the distal portion of the guidewire assembly out from a distal end of the flexible sheath to expose three radiopaque valve-nadir markers of the distal portion disposed approximately 120 degrees from one another about a circle defined by the valve-nadir markers; and
further extending the distal portion out from the sheath towards the aortic valve until the valve-nadir markers stop advancement by reaching respective ones of the aortic valve leaflet nadirs.

2. The method according to claim 1, which further comprises carrying out the guiding and extending steps under fluoroscopy.

3. The method according to claim 1, which further comprises providing the valve-nadir markers at the ends of three respective wire arms each attached at their respective proximal ends to a distal end of the guidewire.

4. A valve-plane-defining catheter, comprising:
a hollow flexible sheath having a distal end and defining a distal opening;
a guidewire assembly having:
a guidewire slidably disposed in the flexible sheath and having a distal end;
three wire arms each having:
a terminating end; and
a proximal end offset from the terminating end and attached to the distal end of the guidewire to dispose the three wire arms approximately 120 degrees from one another about a circle defined by the terminating end of the three wire arms; and
three radiopaque valve-nadir markers disposed respectively at the terminating end of each of the three wire arms, each of the valve-nadir markers being sized to fit within the distal opening.

5. The valve-plane-defining catheter according to claim 4, wherein the proximal end is radially offset from the terminating end.

6. The valve-plane-defining catheter according to claim 4, wherein the proximal end is radially inwardly offset from the terminating end.

7. The valve-plane-defining catheter according to claim 4, wherein the three wire arms each have a different length to position the three valve-nadir markers in line with one another when the three valve-nadir markers are disposed in the distal end of the sheath.
